(19) United States
(12) Patent Application Publication
(10) Pub. No.: US 2010/0016357 A1
(43) Pub. Date: Jan. 21, 2010

(54) Use of Beta-Aminoalcohols for the Treatment of Inflammatory Disorders and Pain

(75) Inventor: Andrew Douglas Baxter, Cambridge (GB)

Correspondence Address: SALIWANCHIK LLOYD & SALIWANCHIK;A PROFESSIONAL ASSOCIATION, PO Box 142950, GAINESVILLE, FL, 32614, US

(73) Assignee: SOSEI R & D LTD., Little Chesterford (GB)

(21) Appl. No.: 12/282,265

(22) PCT Filed: Mar. 9, 2007
(86) PCT No.: PCT/GB2007/000821

§ 371 Date: Sep. 30, 2009

(30) Foreign Application Priority Data
Mar. 9, 2006 (GB) 0604826.8

(51) Int. Cl. A61K 31/445; A61P 11/06; A61P 17/00; A61P 1/00; A61P 19/00; A61P 19/02; A61P 29/00; A61P 1/02; A61P 13/12; A61P 37/00

(52) U.S. Cl. 514/317

(57) ABSTRACT

Compounds that may be used for the treatment or prevention of a condition associated with T-cell proliferation or that is mediated by pro-inflammatory cytokines are of formula (I): wherein at least one of R1, R2 or R3 is not H and each is independently H, alkyl, CF₃, CONH₂, CN, halogen, NH₂, NO₂, NHCHO, NHCONH₂, NHSO₂alkyl, SOMe, SO₂NH₂, Salkyl, or CH₂S0₂alkyl; and R₄ is H or alkyl; or a salt thereof.

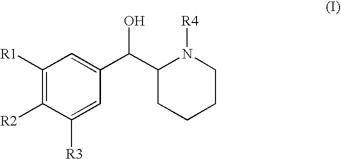

### FIELD OF THE INVENTION

This invention relates to the use of beta-aminoalcohols for the treatment of inflammatory disorders and pain

### BACKGROUND OF THE INVENTION

Immune-driven inflammatory events are a significant cause of many chronic inflammatory diseases where prolonged inflammation causes tissue destruction and results in extensive damage and eventual failure of the effected organ. The cause of these diseases is unknown, so they are often called autoimmune, as they appear to originate from an individual's immune system turning on itself. Conditions include those involving multiple organs, such as systemic lupus erythematosus (SLE) and scleroderma. Other types of autoimmune disease can involve specific tissues or organs such as the musculoskeletal tissue (rheumatoid arthritis, ankylosing spondylitis), gastro-intestinal tract (Crohn's disease and ulcerative colitis), the central nervous system (Alzheimer's, multiple sclerosis, motor neurone disease, Parkinson's disease and chronic fatigue syndrome), pancreatic beta cells (insulin-dependent diabetes mellitus), the adrenal gland (Addison's disease), the kidney (Goodpasture's syndrome, IgA nephropathy, interstitial nephritis), exocrine glands (Sjogren's syndrome and autoimmune pancreatitis) and skin (psoriasis and atopic dermatitis).

In addition, there are chronic inflammatory diseases whose aetiology is more or less known but whose inflammation is also chronic and unremitting. These also exhibit massive tissue/organ destruction and include conditions such as osteoarthritis, periodontal disease, diabetic nephropathy, chronic obstructive pulmonary disease, artherosclerosis, graft versus host disease, chronic pelvic inflammatory disease, endometriosis, chronic hepatitis and tuberculosis. In these diseases, the tissue destruction often damages organ function, resulting in progressive reductions in quality of life and organ failure. These conditions are a major cause of illness in the developing world and are poorly treated by current therapies.

Inflammation of skin structures (dermatitis) is a common set of conditions which include actinic keratosis, acne rosacea, acne vulgaris, allergic contact dermatitis, angioedema, atopic dermatitis, bullous pemiphigoid, cutaneous drug reactions, erythema multiforme, lupus erythrametosus, photodermatitis, psoriasis, psoriatic arthritis, scleroderma and urticaria. These diseases are treated using a wide array of therapies, many of which have very severe side-effects.

Current disease-modifying treatments (if any) for immune-driven conditions include neutralising antibodies, cytotoxics, corticosteroids, immunosuppressants, antihistamines and antimuscarinics. These treatments are often associated with inconvenient routes of administration and severe side-effects, leading to compliance issues. Moreover, certain drug classes are only effective for certain types of inflammatory diseases, e.g. antihistamines for rhinitis.

A known beta-aminoalcohol is rimiterol. Beta-amino alcohols are known to have antihypertensive, vasodilator, sympathomimetic, bronchodilator or cardiostimulant activity through agonism and antagonism at alpha and beta adrenoceptors.

### SUMMARY OF THE INVENTION

Surprisingly, it has been found that certain beta-amino alcohols are inhibitors of cytokines and possess anti-inflammatory properties. According to the present invention, pain or an inflammatory condition, e.g. described above, is treated by the use of a compound of general formula (I)

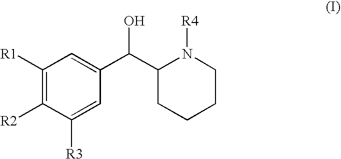

### Wherein

R1, R2 and R3 are independently H, alkyl, CF₃, CONH₂, CN, halogen, NH₂, NO₂, NHCHO, NHCONH₂, NHSO₂alkyl, SOMe, SO₂NH₂, Salkyl or CH₂SO₂alkyl, but are not all H; and

R₄ is H or alkyl;

or a salt thereof.

### DESCRIPTION OF THE INVENTION

Compounds of formula (I) useful for use in the invention include (but are not limited to):
(3,5-dichloro-4-amino-phenyl)-piperidin-2-yl methanol
(3-chloro-phenyl)-piperidin-2-yl methanol
(3,5-dihydroxy-phenyl)-piperidin-2-yl methanol
(3,4-dihydroxy-phenyl)-piperidin-2-yl methanol
(2,3-dihydroxy-phenyl)-piperidin-2-yl methanol
(2,3,4-trihydroxy-phenyl)-piperidin-2-yl methanol
(4-amino-phenyl)-piperidin-2-yl methanol
(3,5-dimethylcarbamoyloxy-phenyl)-piperidin-2-yl methanol
(5,6,7,8-tetrahydro-2-naphthyl)-piperidin-2-yl methanol
(2,5-dimethoxy-phenyl)-piperidin-2-yl methanol
(4-amino-3-cyano-phenyl)-piperidin-2-yl methanol
(2-chloro-phenyl)-piperidin-2-yl methanol
(4-hydroxy-phenyl)-piperidin-2-yl methanol
(3,4-diacetyl-phenyl)-piperidin-2-yl methanol
(3,4-dichloro-phenyl)-piperidin-2-yl methanol
(2,5-dimethoxy-phenyl)-piperidin-2-yl methanol
(4-hydroxy-3-methoxy-phenyl)-piperidin-2-yl methanol
(3-hydroxy-phenyl)-piperidin-2-yl methanol
(4-nitro-phenyl)-piperidin-2-yl methanol
(2-hydroxyquinolin-5-yl)-piperidin-2-yl methanol
(4-hydroxy-3-methanesulphonamide-phenyl)-piperidin-2-yl methanol
(4-phenylmethoxy-3-methanesulphonamide-phenyl)-piperidin-2-yl methanol
(3,4-diphenylmethoxy-phenyl)-piperidin-2-yl methanol
(4-methane-sulphonamide-phenyl)-piperidin-2-yl methanol
(4-hydroxy-sulphonamide-phenyl)-piperidin-2-yl methanol
(2-chloro-4-hydroxy-phenyl)-piperidin-2-yl methanol
(2-fluoro-phenyl)-piperidin-2-yl methanol
(4-fluoro-phenyl)-piperidin-2-yl methanol
(4-bromo-phenyl)-piperidin-2-yl methanol
(4-hydroxy-3-methylsulfonyl-phenyl)-piperidin-2-yl methanol
(3,5-ditertbutylcarbonyloxy-phenyl)-piperidin-2-yl methanol
(3,5-disopropylcarbonyloxy-phenyl)-piperidin-2-yl methanol phenyl-piperidin-2-yl methanol
(3-chloro-4-amino-5-trifluoromethyl-phenyl)-piperidin-2-yl methanol
(naphthalene-2-yl)-piperidin-2-yl methanol
(3,4,5-trihydroxy-phenyl)-piperidin-2-yl methanol
(4-hydroxy-3-hydroxymethyl-phenyl)-piperidin-2-yl methanol
(4-hydroxy-3-methoxy-phenyl)-piperidin-2-yl methanol
(2,5-dimethoxy-phenyl)-piperidin-2-yl methanol
(4-benzyloxy-phenyl)-piperidin-2-yl methanol
(3,4-dibenzyloxy-phenyl)-piperidin-2-yl methanol
(4-methoxy-phenyl)-piperidin-2-yl methanol
(3-methoxy-phenyl)-piperidin-2-yl methanol
(3-methyl-phenyl)-piperidin-2-yl methanol
(4-methyl-phenyl)-piperidin-2-yl methanol
(4-acetamide-3-chloro-phenyl)-piperidin-2-yl methanol
(4-ethoxy-phenyl)-piperidin-2-yl methanol and
(4-nitro-phenyl)-piperidin-2-yl methanol

It is understood that compounds for use in the invention include salts, e.g. the hydrochloride, metabolites and pro-drugs thereof. Compounds for use in the invention are chiral, and it will be understood that this invention includes any diastereomers and enantiomers of (I).

A preferred diastereomer or enantiomer of (I) has little or no activity at the α or β adrenoceptors. This activity may be determined by use of the appropriate in vitro assay. Particularly preferred compounds include erythro-(S)-4-amino-3,5-dichlorophenyl-(R)-piperidin-2-yl-methanol, threo-(S)-4-amino-3,5-dichlorophenyl-(S)-piperidin-2-yl-methanol and erythro(S)-4-amino-3,5-dichlorophenyl-(R)-piperidin-2-yl methanol.

The compounds of formula (I) according to the invention are used to treat inflammatory diseases including, but not exclusive to, autoimmune diseases involving multiple organs, such as systemic lupus erythematosus (SLE) and scleroderma, specific tissues or organs such as the musculoskeletal tissue (rheumatoid arthritis, ankylosing spondylitis), gastro-intestinal tract (Crohn's disease and ulcerative colitis), the central nervous system (Alzheimer's, multiple sclerosis, motor neurone disease, Parkinson's disease and chronic fatigue syndrome), pancreatic beta cells (insulin-dependent diabetes mellitus), the adrenal gland (Addison's disease), the kidney (Goodpasture's syndrome, IgA nephropathy, interstitial nephritis) exocrine glands (Sjogren's syndrome and autoimmune pancreatitis) and skin (psoriasis and atopic dermatitis), chronic inflammatory diseases such as osteoarthritis, periodontal disease, diabetic nephropathy, chronic obstructive pulmonary disease, artherosclerosis, graft versus host disease, chronic pelvic inflammatory disease, endometriosis, chronic hepatitis and tuberculosis, IgE mediated (Type I) hypersensitivities such as rhinitis, asthma, anaphylaxis and dermatitis. Dermatitis conditions include actinic keratosis, acne rosacea, acne vulgaris, allergic contact dermatitis, angioedema, atopic dermatitis, bullous pemiphigoid, cutaneous drug reactions, erythema multiforme, lupus erythrametosus, photodermatitis, psoriasis, psoriatic arthritis, scleroderma and urticaria. Conditions of the eye, such as diabetic retinopathy, macular degeneration, choroidal neovascular membrane, cystoid macular edema, epi-retinal membrane, macular hole, dry eye, uveitis and conjunctivitis, may also be treated.

These compounds may be used according to the invention when the patient is also administered or in combination with another therapeutic agent selected from corticosteroids (examples including cortisol, cortisone, hydrocortisone, dihydrocortisone, fludrocortisone, prednisone, prednisolone, deflazacort, flunisolide, beconase, methylprednisolone, triamcinolone, betamethasone, and dexamethasone), disease modifying anti-rheumatic drugs (DMARDs) (examples including azulfidine, aurothiomalate, bucillamine, chlorambucil, cyclophosphamide, leflunomide, methotrexate, mizoribine, penicillamine and sulphasalazine), immunosuppressants (examples including azathioprine, cyclosporin, mycophenolate), COX inhibitors (examples including aceclofenac, acemetacin, alcofenac, alminoprofen, aloxipirin, amfenac, aminophenazone, antraphenine, aspirin, azapropazone, benorilate, benoxaprofen, benzydamine, butibufen, celecoxib, chlorthenoxacine, choline salicylate, chlometacin, dexketoprofen, diclofenac, diflunisal, emorfazone, epirizole, etodolac, feclobuzone, felbinac, fenbufen, fenclofenac, flurbiprofen, glafenine, hydroxylethyl salicylate, ibuprofen, indometacin, indoprofen, ketoprofen, ketorolac, lactyl phenetidin, loxoprofen, mefenamic acid, metamizole, mofebutazone, mofezolac, nabumetone, naproxen, nifenazone, oxametacin, phenacetin, pipebuzone, pranoprofen, propyphenazone, proquazone, rofecoxib, salicylamide, salsalate, sulindac, suprofen, tiaramide, tinoridine, tolfenamic acid, zomepirac) neutralising antibodies (examples including etanercept and infliximab), antibiotics (examples including doxycycline and minocycline).

Compounds of formula (I) exhibit analgesic activity in animal models. The activity of these compounds may be determined by the use of the appropriate in vivo assay.

This invention also relates to a method of treatment for patients (including man and/or mammalian animals raised in the dairy, meat or fur industries or as pets) suffering from chronic, acute or neuropathic pain; and more specifically, a method of treatment involving the administration of the analgesic of formula (I) as the active constituent.

Accordingly, the compounds of formula (I) can be used inter alia in the treatment of pain conditions such as acute and chronic pain (as well as, but not limited to, pain associated with cancer, surgery, arthritis, dental surgery, trauma, musculo-skeletal injury or disease, visceral diseases) and migraine headache. Additionally the painful conditions can be neuropathic; examples of such conditions are post-herpetic neuralgia, diabetic neuropathy, drug-induced neuropathy, HIV-mediated neuropathy, sympathetic reflex dystrophy or causalgia, fibromyalgia, myofacial pain, entrapment neuropathy, phantom limb pain and trigeminal neuralgia. Neuropathic conditions include central pain related to stroke, multiple sclerosis, spinal cord injury, arachnoiditis, neoplasms, syringomyelia, Parkinson's disease and epilepsia.

It will often be advantageous to use compounds of formula (I) in combination with another drug used for pain therapy. Such another drug may be an opiate or a non-opiate such as baclofen. Especially for the treatment of neuropathic pain, coadministration with gabapentin is preferred. Other compounds that may be used include acetaminophen, a non-steroidal anti-inflammatory drug, a narcotic analgesic, a local anaesthetic, an NMDA antagonist, a neuroleptic agent, an anti-convulsant, an anti-spasmodic, an anti-depressant or a muscle relaxant.

Any suitable route of administration can be used. For example, any of oral, topical, parenteral, ocular, rectal, vaginal, inhalation, buccal, sublingual and intranasal delivery routes may be suitable. The dose of the active agent will depend on the nature and degree of the condition, the age and condition of the patient and other factors known to those skilled in the art. A typical dose is from 0.1, e.g. 10 to 100, mg given one to three times per day.

Compounds for use in the invention may be prepared by a multi-step synthetic procedure, as shown in the following Scheme.

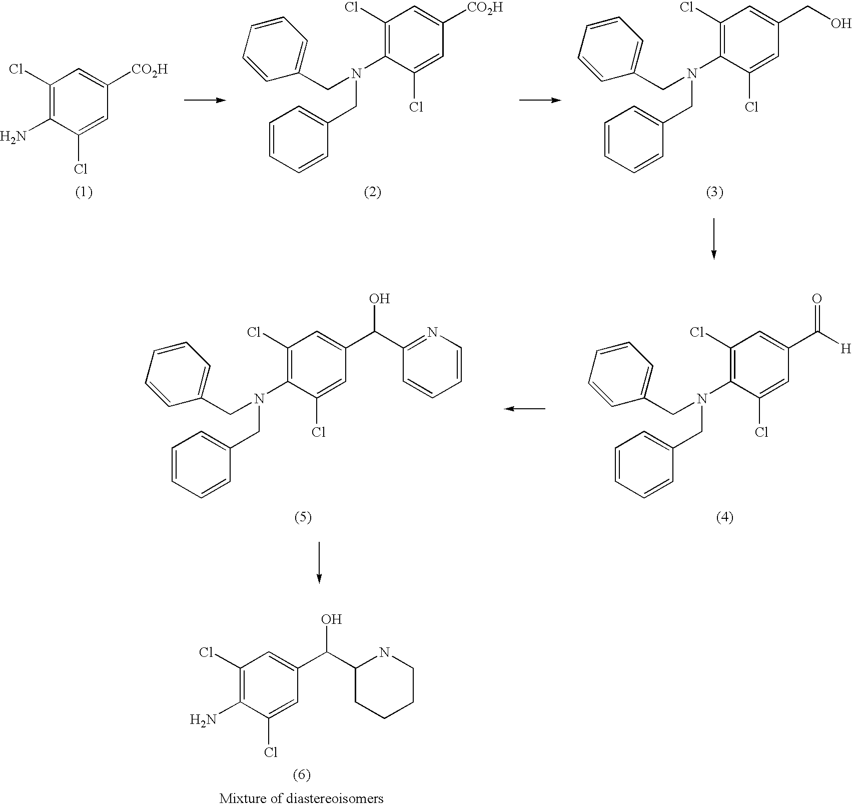

The synthesis proceeds by reduction of the carboxylic acid group of a substituted aromatic ring using a suitable reagent, followed by its oxidation through to the corresponding aldehyde, which can then be reacted with a halopyridine moiety. Saturation of this ring is facilitated by a hydrogenation procedure utilising a suitable catalyst to give the target molecule as a racemic mixture. As will be apparent to one of ordinary skill in the art, functional groups present in the molecules can be protected and deprotected, as needed.

Isolation of the separate diastereomeric pairs can be achieved, either directly via a purification technique such as trituration, or indirectly, for example by initial conversion to an intermediate ester which can then be purified by trituration/similar method and then hydrolysed back to the parent compound. Each of the diastereomeric pairs can then be further separated into their pure isomeric components via CHIRAL HPLC.

The following synthesis illustrates the preparation of compounds for use in the invention.

### 3,5-Dichloro-4-dibenzylaminobenzoic acid (2)

4-Amino-3,5-dichlorobenzoic acid (1) (25.0 g, 0.121 mol) was dissolved in a mixture of THF (250 mL) and DMF (50 mL) under a nitrogen atmosphere. Benzyl bromide (62.3 g, 0.364 mol) was added at 0° C. and with stirring, sodium hydride (60% w/w in mineral oil, 19.4 g, 0.485 mol) was added portionwise over 10 mins. Hydrogen gas was rapidly evolved and was vented to atmosphere. After NaH addition was complete, the suspension was warmed to RT and stirring continued for 16 h. After this time, the suspension was cooled to 0° C. and H₂O (100 mL) added. The mixture was then further diluted with an aqueous 1 M HCl solution (250 mL). The aqueous layer was extracted into ethyl acetate, dried (MgSO₄), filtered and partially concentrated in vacuo. Toluene (100 mL) was added and the solution azeotroped to give a pale yellow solid. Trituration with heptanes and then filtration under suction gave 3,5-dichloro-4-dibenzylaminobenzoic acid (2) (41.3 g, 88%).

δ_{H}(DMSO-d6; 250 MHz) 4.31 (4H, s, CH₂ Ph), 7.33-7.25 (10H, m, ArH), 7.81 (2H, s, ArH), missing COOH.

### (3,5-Dichloro-4-dibenzylaminophenyl)methanol (3)

3,5-Dichloro-4-dibenzylaminobenzoic acid (2) (40.57 g, 0.105 mol) was dissolved in THF (390 mL) then cooled to 0° C. before dropwise addition of borane tetrahydrofuran complex (1 M in THF, 210 mL, 0.21 mol) over 20 minutes. After complete addition, the solution was warmed to RT and stirring continued for 5 hours or until complete by TLC. MeOH (100 mL) was added slowly via dropping funnel and once gas evolution had ceased, the solution was concentrated in vacuo to provide colourless oil. The crude oil could be further purified on silica gel (eluant 4:1 heptanes:ethyl acetate) or used as crude in the following reaction (37.3 g).

δ_{H}(CDCl₃; 250 MHz) 4.26 (4H, s, CH₂ Ph), 4.59 (2H, s, CH₂ OH), 7.19-7.37 (12H, m, ArH), missing OH.

### 3,5-Dichloro-4-dibenzylaminobenzaldehyde (4)

Crude (3,5-dichloro-4-dibenzylaminophenyl)methanol (3) prepared as above (37.3 g, 0.105 mol) was dissolved in dichloromethane (400 mL) and heated to reflux under nitrogen. Activated manganese dioxide (MnO₂) (23.9 g, 0.275 mol) was added in one portion and heating continued for 3 h. After this time, further MnO₂ (23.9 g, 0.275 mol) was added and the mixture heated overnight. Analysis by TLC showed incomplete reaction therefore further portions of MnO₂ were added (3×23.0 g) until the reaction was deemed complete. The suspension was filtered through celite under suction then the filter cake was washed with THF (500 mL) until colourless. The orange filtrate solution was concentrated in vacuo then trituration in cold heptanes provided 3,5-dichloro-4-dibenzylaminobenzaldehyde (4) as a pale yellow solid in two batches (26.4 g, 78%).

δ_{H}(CDCl₃; 250 MHz) 4.32 (4H, s, CH₂ Ph), 7.24-7.40 (10H, m, ArH), 7.77 (2H, s, ArH), 9.84 (1H, s, CHO).

### (3,5-Dchloro-4-dibenzylaminophenyl)pyridin-2-ylmethanol (5)

2-Iodopyridine (13.53 g, 0.066 mol) was dissolved in anhydrous THF (200 mL) under nitrogen at 0° C. Ethylmagnesium bromide solution (0.79 M in THF, 100 mL, 0.079 mol) was added dropwise via dropping funnel over 1 h then the mixture was warmed to RT. 3,5-Dichloro-4-dibenzylaminobenzaldehyde (4) (26.4 g, 0.071 mol) in THF (118 mL) was added dropwise over 15 minutes with a slight exotherm noted. After a further 2 h, the reaction was complete by TLC. The reaction was quenched by dropwise addition of 2 M HCl (100 mL) and extracted into ethyl acetate. The organic layer was separated, washed with 2 M NaOH (aq.), dried (MgSO₄) and filtered. The organic solution was evaporated to provide a yellow oil which was purified by chromatography on silica gel (4:1 heptanes:EtOAc then 1:1 heptanes:EtOAc) to provide a colourless oil which was found to be (3,5-dichloro-4-dibenzylamino-phenyl)-pyridin-2-yl-methanol (5) (19.56 g, 66%).

δ_{H}(CDCl₃; 250 MHz) 4.23 (4H, s, CH₂ Ph), 5.66 (1H, s, CHOH), 7.13 (1H, d, J7.7, PyrH), 7.15-7.34 (13H, m, ArH), 7.71 (1H, dd, J1.7, 7.7, PyrH), 8.59 (1H, d, J4.8, PyrH), missing OH.

### Erythro-(3,5-dichloro-4-amino-phenyl)-piperidin-2-yl-methanol (6)

A 2 L Parr hydrogenator was charged with (3,5-dichloro-4-dibenzylamino-phenyl)-pyridin-2-yl-methanol (5) (19.37 g, 0.043 mol) dissolved in EtOH (200 mL). A solution of HCl in MeOH (1.25 M, 76 mL, 0.095 mol) was introduced and finally PtO₂ (3.1 g, 0.013 mol) was added. The suspension was pressurised with hydrogen gas to 50 p.s.i. at RT. Rapid uptake of hydrogen was noted and after one hydrogen gas recharge, the reaction was complete by ¹H-NMR. The system was depressurised and the ethanolic suspension filtered through celite under suction. The filtrate was concentrated in vacuo and then purified on silica gel (CH₂Cl₂ then 95:5:1 CH₂Cl₂:MeOH:Et₃N then 95:7:1 CH₂Cl₂:MeOH:Et₃N) to provide (3,5-dichloro-4-amino-phenyl)-piperidin-2-yl-methanol as an inseparable mixture of diastereomers (10.6 g, 89%) (6). The mixture was triturated in ice-cold acetone (25 mL), which caused crystallisation of a white solid. This white solid was isolated by filtration and confirmed as pure erythro-(3,5-dichloro-4-amino-phenyl)-(piperidin-2-yl-methanol by ¹H-NMR (1.65 g, 14%). The filtrate solution was concentrated in vacuo and found to contain a mixture of diastereomers (6) (6.73 g).

Erythro δ_{H}(CDCl₃; 250 MHz) 1.10-1.47 (4H, m), 1.47-1.68 (2H, m), 2.00-2.30 (2H, m), 2.55-2.78 (2H, m), 3.10 (1H, app. d, J11.8), 4.42 (2H, s), 4.49 (1H, d, J4.9, CHOH), 7.19 (2H, s, ArH).

The erythro diastereomeric pair was separated by preparative CHIRAL HPLC, using a 260×50 mm CHIRALPAK® AD 20 μm column, a mobile phase of 80 n-heptane/20 ethanol/0.1 diethylamine (v/v/v), a flow rate of 120 ml/min and a UV detection wavelength of 300 nm at ambient temperature.

### Erythro-(3,5-dichloro-4-amino-phenyl)-piperidin-2-yl-methanol (7)

This compound was obtained as 600 mg of the first eluting isomer, isolated as an oil.

Retention time 6.5 min
HPLC analysis (area % at 230 nm)>99.5
Enantiomeric excess (%)>99.5

### (Erythro)-(3,5-dichloro-4-amino-phenyl)-piperidin-2-yl-methanol (8)

This compound was obtained as 600 mg of the second eluting isomer, isolated as an oil.

Retention time 9.2 min
HPLC analysis (area % at 230 nm)>99.0
Enantiomeric excess (%)>99.0

### Threo-2-(3,5-Dichloro-4-aminophenyl)hydroxymethyl)piperidine-1-carboxylic acid tert-butyl ester

The diastereomeric mixture of (3,5-dichloro-4-aminophenyl)piperidin-2-ylmethanol (6.73 g, 0.024 mol) was dissolved in dichloromethane (44 mL) and then triethylamine (6.8 mL, 0.049 mol) was added. The solution was cooled to 0° C. under a nitrogen atmosphere then di-tert-butyl dicarbonate (5.81 g, 0.027 mol) was added portionwise. The resulting mixture was stirred for 6 h, until TLC showed consumption of starting material. The reaction was quenched with aq. 1M NaOH and extracted into dichloromethane (2×50 mL). The organic extracts were dried (MgSO₄), filtered and concentrated in vacuo to provide viscous, colourless oil, which was found to be a diastereomeric mixture of 2-[(3,5-dichloro-4-amino-phenyl)-hydroxy-methyl]-piperidine-1-carboxylic acid tert-butyl ester (9.24 g, 99%). The mixture was triturated in ice-cold heptanes (25 mL) and a white solid crystallised. The white solid formed was isolated by filtration and found to be pure threo-2-(3,5-dichloro-4-amino-phenyl)-hydroxy-methyl)-piperidine-1-carboxylic acid tert-butyl ester by ¹H-NMR (2.46 g, 27%).

Threo δ_{H}(CDCl₃; 250 MHz) 1.30 (9H, s, C(CH₃)₃), 1.35-1.80 (6H, m), 2.03 (1H, d, J11.5), 2.74 (1H, app. t, J12.3), 3.85-3.95 (1H, m), 4.13-4.25 (1H, m), 4.41 (2H, s), 4.79 (1H, d, J8.1, CHOH), 7.20 (2H, s, ArH).

### Threo-(3,5-Dichloro-4-amino-phenyl)-piperidin-2-yl-methanol (9)

Threo-2-(3,5-Dichloro-4-aminophenyl)hydroxymethyl)piperidine-1-carboxylic acid tert-butyl ester (1.50 g, 0.004 mol) was suspended in dichloromethane (25 mL) at 0° C. under a nitrogen atmosphere. Trifluoroacetic acid (0.68 mL, 0.009 mol) was added dropwise causing the suspension to dissolve. After 3 h, further trifluoroacetic acid (0.25 mL, 0.0032 mol) was added and the solution stirred overnight at RT. The reaction was quenched with aq. 1M NaOH and extracted into dichloromethane (2×25 mL). The organic extracts were dried (MgSO₄), filtered and concentrated in vacuo to give an off-white solid. Trituration with ice-cold heptanes (10 mL) gave a white solid, which was found to be pure Threo-(3,5-dichloro-4-amino-phenyl)-piperidin-2-yl-methanol by ¹H-NMR (0.893 g, 79%).

Threo δ_{H}(CDCl₃; 250 MHz) 1.10-1.80 (7H, m), 1.94 (1H, app. d, J8.3), 2.83 (1H, app. t, J11.6), 3.29-3.47 (1H, m), 3.91 (1H, dd, J1.2, 11.6), 4.30-4.70 (2H, br. s, NH₂), 4.83 (1H, d, J7.0, CHOH), 7.19 (2H, s, ArH).

The threo diastereomeric pair was separated by preparative CHIRAL HPLC, using a 250×20 mm CHIRALPAK® AS-H 5 μm column, a mobile phase of 80 CO₂/20 methanol+1% diethylamine (v/v), a flow rate of 60 ml/min and a UV detection wavelength of 250 nm at a temperature of 30° C. and an outlet pressure of 150 bar.

### Threo-(3,5-dichloro-4-amino-phenyl)-piperidin-2-yl-methanol (10)

This compound was obtained as 259 mg of the first eluting isomer, isolated as an oil.

Retention time 16.8 min
HPLC analysis (area % at 250 nm) 97.2
Enantiomeric excess (%) 99.9

### Threo-(3,5-Dichloro-4-amino-phenyl)-piperidin-2-yl-methanol (11)

This compound was obtained as 282 mg of the second eluting isomer, isolated as an oil.

Retention time 14.6 min
HPLC analysis (area % at 250 nm) 94.7
Enantiomeric excess (%) 98.8

The following Assays illustrate the invention.

### Beta2 Agonism Functional Assay

Guinea-pig trachea ring preparations were suspended in Kreb's solution containing indomethacin. After 15 minutes stabilisation, the preparations were repeated contracted using carbachol and simultaneously treated with increasing cumulative doses test compounds (0.1 nM to 0.1 μM). Beta2 agonism for each test compound was determined by its dose-dependant inhibition of carbachol-stimulated tracheal muscle twitch.

Compounds (7), (8), (10) and (11) were poor, beta2 agonists. The IC50 values for all four compounds were >2 μM, with three of the four compounds having values >20 μM.

### LPS Mouse Assay

7 week old Balb C ByJ mice (24-28 g) were administered, either by i.p. (5 ml/kg) or oral (10 ml/kg) administration, with vehicle or test article. 30 minutes later these animals were challenged with an intraperitoneal injection of 1 mg/kg LPS. 2 hours after LPS challenge blood samples were collected under light isoflurane anaesthesia into normal tubes by retro-orbital puncture. Samples were allowed to clot at room temperature and then spun at 6000 g for 3 min at 4° C. Serum was stored at −20° C. until use. Serum TNFα and IL-10 levels were analysed in duplicate by ELISA technique.

Compounds (7), (8), (10) and (11) all had effects on LPS-induced TNFα and IL1β cytokine production in mice. Two compounds effectively inhibited all cytokines, at all doses.

1. A method for the treatment or prevention of an inflammatory condition or pain, wherein said method comprises administering, to a subject in need of such treatment or prevention, a compound of formula (I) 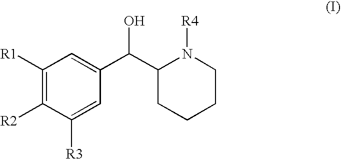
wherein,
R1, R2 and R3 are independently H, alkyl, CF₃, CONH₂, CN, halogen, NH₂, NO₂, NHCHO, NHCONH₂, NHSO₂alkyl, SOMe, SO₂NH₂, Salkyl or CH₂SO₂alkyl, but are not all H; and
R₄ is H or alkyl;
or a salt thereof.

2. The method according to claim 1, wherein the condition is a chronic degenerative disease.

3. The method according to claim 1, wherein the condition is a chronic demyelinating disease.

4. The method according to claim 1, wherein the condition is a respiratory disease.

5. The method according to claim 1, wherein the condition is an inflammatory bowel disease.

6. The method according to claim 1, wherein the condition is a dermatological condition.

7. The method according to claim 1, wherein the condition is a dental disease.

8. The method according to claim 1, wherein the condition is diabetic nephropathy, lupus nephritis, IgA nephropathy or glomerulonephritis.

9. The method according to claim 1, wherein the condition is systemic lupus erythematosus.

10. The method according to claim 1, wherein the condition is graft vs host disease.

11. The method according to claim 1, wherein the condition is a pain condition.

12. The method according to claim 11, wherein the pain condition is chronic pain.

13. The method according to claim 11, wherein the pain condition is acute pain.

14. The method according to claim 11, wherein the pain condition is neuropathic pain.

15. The method according to claim 1, wherein the condition is an ophthalmic condition.

16. The method according to claim 15, wherein the ophthalmic condition is age related macular degeneration.

17. The method according to claim 15, wherein the ophthalmic condition is diabetic retinopathy.

18. The method according to claim 15, wherein the ophthalmic condition is choroidal neovascular membrane, cystoid macular edema, epi-retinal membrane or macular hole.

19. The method according to claim 15, wherein the ophthalmic condition is dry eye.

20. The method according to claim 15, wherein the ophthalmic condition is uveitis.

21. The method according to claim 1, wherein R1, R2 and R3 are independently CF₃, CONH₂, CN, halogen or NH₂.

22. The method according to claim 1, wherein the compound is erythro-(S)-4-amino-3,5-dichlorophenyl-(R)-piperidin-2-yl-methanol, threo-(S)-4-amino-3,5-dichlorophenyl-(S)-piperidin-2-yl-methanol or erythro-(S)-4-amino-3,5-dichlorophenyl-(R)-piperidin-2-yl-methanol.

23. The method according to claim 1, wherein the subject is also administered another therapeutic agent selected from corticosteroids, cytotoxics, antibiotics, immunosupressants, non-steroidal anti-inflammatory drug, narcotic analgesics, local anaesthetics, NMDA antagonists, neuroleptics, anti-convulsants, anti-spasmodics, anti-depressants and muscle relaxants.

24. The method according to claim 23, wherein the compound (I) and said another agent are provided in combination.
